(19) United States
(12) Patent Application Publication
(10) Pub. No.: US 2011/0256147 A1
(43) Pub. Date: Oct. 20, 2011

(54) DIAGNOSTIC AND TREATMENT METHODS FOR CANCER BASED ON IMMUNE INHIBITORS

(75) Inventors: Mathias Oelke, Baltimore, MD (US); Jonathan Schneck, Baltimore, MD (US); Tonya Webb, Baltimore, MD (US)

(73) Assignee: THE JOHNS HOPKINS UNIVERSITY, Baltimore, MD (US)

(21) Appl. No.: 13/132,234

(22) PCT Filed: Dec. 1, 2009
(86) PCT No.: PCT/US2009/066262

§ 371 Date: Jul. 6, 2011

Related U.S. Application Data
(60) Provisional application No. 61/118,817, filed on Dec. 1, 2008.
(60) Provisional application No. 61/175,977, filed on May 6, 2009.

(51) Int. Cl. A61K 39/395; C12Q 1/02; G01N 33/53; A61P 11/06; A61K 31/702; A61P 35/00; A61P 37/06; A61P 37/08; G01N 33/566; A61K 31/713

(52) U.S. Cl. 424/155.1; 435/7.21; 435/7.24; 435/29; 435/7.93; 424/174.1; 514/44 A; 514/54

(57) ABSTRACT

Methods of diagnosis and treatment of malignant tumors, in particular ovarian tumors, using GD3 and GD3 inhibitors. Also provided are methods of modulating the immune system of a mammal by the administration of a GD3 and GD3 inhibitors.

The work leading to the invention described and claimed herein was carried out using funds from the United States Department of Health and Human Services, grants no. NIH AI 44129, CA 108835, & P01 AI072677. The U.S. government has certain rights in the invention.

### BACKGROUND OF THE INVENTION

1. Field of the Invention

The invention relates to methods of diagnosis and treatment of solid tumors, in particular ovarian tumors using GD3 and GD3 inhibitors. The invention also relates to a method of modulating the immune system of a mammal by the administration of GD3 or GD3 inhibitors. 2. Background Information

Epithelial ovarian cancer is the leading cause of gynecological cancer deaths worldwide (1). Currently, the 5 year survival rate of patients with ovarian cancer is less than 30%. This is a direct result of the majority of cases being diagnosed at an advanced stage. Ovarian cancer is a multifarious disease in that there is a successive accumulation of multiple molecular alterations in both the cells undergoing neoplastic transformation and the host cells. Therefore, each tumor tends to be molecularly distinct, increasing the difficulty of identifying a molecular target with prognostic potential.

In order to maintain normal physiological homeostasis, the host's immune system must be able to recognize neoplastic transformation and destroy transformed cells. This is a layered process, where the first recognition events are relatively non-specific followed by the activation of the adaptive immune system, ultimately leading to sterilizing immunity. One of the earliest pathways in immune activation is the presentation of phospho/glycolipid antigens by CD1d molecules to a unique subpopulation of T cells called natural killer T (NKT) cells. NKT cells are primed cells that have large reservoirs of cytokines such as IFN-γ which can, if appropriately activated these cytokines are released, initiating the development of a robust adaptive immune response.

We have discovered that treatment of CD1 d-expressing cells with ascites from ovarian cancer patients, particularly those with high-grade serous carcinoma, abrogated their ability to activate both canonical and noncanonical NKT cells. Many studies have sought to characterize the adaptive T cell immune response in epithelial ovarian cancer (EOC); however, mechanisms of immune evasion by ovarian cancers, specifically affecting the NKT cell/CD1 d system remain to be elucidated.

Ovarian cancer associated ascites contains cellular components of the immune system such as lymphocytes and regulatory factors such as cytokines. It has been reported that patients with advanced ovarian cancer have higher levels of gangliosides in their plasma and ascites compared to plasma ganglioside levels of controls (2) furthermore it has been shown that abnormal ganglioside expression is strongly associated with the malignancy of cancer cells. Cancer patients that have high circulating ganglioside levels at the time of clinical diagnosis exhibit a faster rate of disease progression and a decreased rate of survival (3).

This application claims priority to U.S. provisional application no. 61/118,817, filed Dec. 1, 2008 and U.S. provisional application no. 61/175,977, filed May 6, 2009, each of which is hereby incorporated by reference.

### BRIEF DESCRIPTION OF THE DRAWINGS

FIG. 1. Treatment with ascites from ovarian cancer patient inhibits CD1d-mediated activation of NKT cells. (A) LCD1dwt cells were treated with media or ascites from the indicated patients for 4 h, washed extensively and then co-cultured with a panel of NKT cell hybridomas, DN32.D3, N37-1A12, N38-3C3, N38-2C12, and N38-2H4. IL-2 was measured by standard cytokine ELISA, and data is shown as percent inhibition, normalized to cells treated with culture media as 100% or the maximum level of stimulation. The results are representative of more than 15 experiments, in which 3-12 patient samples were tested in each experiment and each sample repeated at least twice. (B) The ascites treated LCD1d1wt cells were co-cultured with the Vα14⁺ NKT cell hybridomas, DN32.D3, N38-2H4, (C) primary mouse NKT cells, or (D) primary human NKT cells and recognition of CD1d was assessed by measuring cytokine production in the supernatants by ELISA. NKT cells were co-cultured in media alone or with L cells transfected with vector alone as controls. Spontaneous cytokine release and background levels of cytokine production (as measured by co-culturing NKT cells with L cells containing vector alone) were subtracted before calculating percent inhibition.

FIG. 2. Treatment with serum from ovarian cancer patient does not inhibit CD1d-mediated antigen presentation. LCD1dwt cells were treated with 1 ml media containing 5% FBS, FBS, human AB serum or serum from the indicated patients for 3-4 h, washed extensively and then co-cultured with N37-1A12 and N38-3C3 NKT cell hybridomas. IL-2 was measured by standard cytokine ELISA, and data is shown as percent control, normalized to cells treated with culture media as 100% or the maximum level of stimulation.

FIG. 3. (A) Generation of CD1d-based aAPC. aAPC were made by coupling CD1d-Ig and anti-CD28 Abs to magnetic beads. In this system, CD1d-Ig is used to provide the cognate antigen specific signal through the TCR. (B) Ascites treatment of α-GalCer loaded aAPC inhibits antigen presentation by CD1d molecules. α-GalCer loaded aAPC were incubated for 4 h with either media or ascites from patients (a representative patient [OC-47] is shown). The aAPC (1×10⁵ cells/well) were washed extensively and then co-cultured with the Vα14⁺ NKT cell hybridomas, DN32.D3 (5×10⁴) or N38-2H4 (1×10⁵). NKT cell recognition of CD1d1 was assessed by measuring IL-2 production in the supernatants by ELISA.

FIG. 4. Treatment with ovarian cancer associated ascites has no effect on HLA-A2 mediated antigen presentation. (A) T2 cells were pulsed with CMV peptide, treated with media (CMV-T2) or ascites (OC47 & OC57) for 4 h, washed and cocultured with CMV-specific CD8⁺T cells (1×10⁵ cells/well) at the indicated ratios. IFN-γ released was measured by ELISA. (B) LCD1d1wt cells were treated with ascites and then co-cultured with the Vα14⁺ NKT cell hybridomas, DN32.D3, N38-2H4, and the Vα14⁻ NKT cell hybridoma, N37-1A12. NKT cell recognition of CD1d was assessed by measuring IL-2 production in the supernatants by ELISA. The results are representative of three experiments with similar results.

FIG. 5. NKT cell activation by CD1d1 molecules is inhibited by treatment with ascites. LCD1d1wt cells were incubated for 4 h in the presence or absence of the indicated concentrations of ascites from patient OC-47. The cells were then co-cultured with the Vα14⁺ NKT cell hybridomas, (A) DN32.D3, (B) N38-2H4, (C) Vα14⁻ NKT cell hybridoma, N37-1A12, and the Vα14⁺ NKT cell hybridoma (D) N38-2C12. NKT cell recognition of CD1d1 was assessed by measuring IL-2 production in the supernatants by ELISA. The results are representative of two experiments with patient OC-47 ascites. Similar results were obtained when the experiment was performed with ascites from another patient (OC-40).

FIG. 6. Tumor Ascites Inhibits CD1d-mediated NKT cell activation. (A) LCD1 cells were treated with media or ascites for 4 h, then washed extensively and cocultured with a panel of NKT cell hybridomas. IL-2 was measured, as an indication of NKT cell activation, by standard cytokine ELISA. (B) NKT cell recognition of CD1d1 molecules is rapidly inhibited by treatment with ascites. LCD1d1wt cells were incubated for the indicated time periods with ascites. The cells were then co-cultured with NKT cell hybridomas. The results are representative of two experiments with patient OC-66 ascites. (C) Cultured supernatant from ascites-derived cells is inhibitory. LCD1d1wt cells were incubated for 4 h with fresh cell culture media, ascites, or conditioned media from ascites-derived cells from patient OC-95. The cells were then co-cultured NKT cell hybridomas. NKT cell recognition of CD was assessed by measuring IL-2 production in the supernatants by ELISA. The results are representative of two experiments with patient OC-95 ascites.

FIG. 7. The Polar Lipid Fraction of Ascites is rich in gangliosides. The polar lipid fraction was isolated from the ascites of the indicated ovarian cancer patients. Methanol then chloroform were added to give chloroform-methanol-water (4:8:3), and the samples were extracted. LCD1 cells were treated with ascites or the polar lipid fraction of the ascites for 4 h, then washed extensively and cocultured with a panel of NKT cell hybridomas. (A) IL-2 was measured, or GM-CSF (B) as an indication of NKT cell activation, by standard cytokine ELISA. (C) TLC of Polar Lipid Fraction. The extracted lipids were analyzed by thin-layer chromatography (TLC) (HPTLC Silica Gel 60; Merck, Darmstadt, Germany) with chloroform-methanol-0.25% aqueous KCl (60:35:8) as running solvent. Gangliosides were detected with a resorcinol—HCl Cu2+ reagent. Bovine brain gangliosides were used as standards. (G) A schematic of ganglioside biosynthesis.

FIG. 8. Ganglioside treatment can alter CD1d-mediated NKT cell activation. LCD1dwt cells were treated with (A) GD3 (B) GT1b or (C) GD2 at the indicated concentrations for 4 h, washed extensively and cocultured with NKT cell hybridomas, DN32.D3, N37-1A12, and N38-3C3 overnight. The vehicle for GD3 was chloroform methanol, and methanol for GT1b and GD2. (D) LCD1dwt cells were treated with the indicated gangliosides (1 μg/ml) for 4 h, washed extensively and cocultured with NKT cell hybridomas, the vehicle for all was methanol (E) Treatment of α-GalCer loaded CD1d-Ig aAPC with GD3 inhibits antigen presentation. α-GalCer—loaded aAPC were incubated for 4 h with either media, ascites, GD3, or GD2 (5 μg/ml). The aAPC were washed extensively and then cocultured with the Vα14+ NKT cell hybridoma, N38-3C3. NKT cell recognition of CD1d was assessed by measuring IL-2 production in the supernatants by ELISA. (F) The Addition of anti-GD3 mAb to tumor associated ascites can restore NKT cell recognition of CD1d molecules. LCD1dwt cells were treated with ascites (OC-91) or the ascites pretreated with an antibody specific for GD3 for 4 h, then washed extensively and cocultured with a panel of NKT cell hybridomas. IL-2 was measured, as an indication of NKT cell activation, by standard cytokine ELISA. (G) A schematic of ganglioside biosynthesis is listed below.

FIG. 9. GD3 can compete with PE for binding to CD1d. (A) Nunc MaxiSorp flat-bottom 96 well plates were coated with goat anti-mouse IgG Fc gamma antibody and the plates were washed and blocked. Then the wells were coated with either mCD1d-IgG1 dimer (A) or hCD1d-Ig dimer (B), after washing, serially-diluted lipid were added into the wells in the presence of 2 μg/ml Biotinyl PE. Then, the plates were washed and the amounts of CD 1 d-Ig-Biotinyl PE complex was detected by adding HRP-labeled Avidin. (C) GD3 loaded CD 1 d dimeric molecules do not stain NKT cells. Mouse CD-1-d-Ig dimer (5 μg/mL) was incubated with α-GalCer (2 μg/ml), ascites (diluted 5-fold in PBS), or GD3 (2 μg/ml) to load the lipid onto the dimer, then used to stain NKT cells. The cells were washed and incubated with phycoerythrin-labeled rat anti-mouse IgG1 antibody (A85-1). After washing, the stained cells were analyzed by flow cytometry.

### DESCRIPTION OF THE INVENTION

The present inventors analyzed the lipid fraction of ovarian cancer associated ascites and identified the ganglioside GD3 as a factor that significantly inhibited the activation of NKT cells through replacement of the natural CD1d restricted ligands. Their studies demonstrated that antigen processing was not required because CD-1-d-Ig dimers loaded with α-GalCer were no longer recognized by NKT cells following treatment with ascites or GD3. Furthermore, GD3 bound with high affinity to CD1d supporting the conclusion that ganglioside shedding may be an early mechanism of immune evasion.

Accordingly, in one aspect, the invention provides a method of diagnosis of a malignant tumor comprising measuring the level of GD3 in a biological sample obtained from a mammal, e.g. a human, and comparing the level to a control value, wherein an increase in the level of GD3 compared to the control value is indicative of a likelihood that the mammal suffers from a malignant tumor. Tumors to be diagnosed include those that are known to be of viral origin, as well as those that are not of viral origin. The compositions and methods of the invention are expected to be particularly useful in the diagnosis of solid tumors, such as ovarian cancer. Methods of measuring GD3 are known to those of skill in the art, and examples are described herein below. For example, GD3 is expected to be detected in ELISA or ELISA-like assays. Also provided is a method of diagnosis of a malignant tumor comprising measuring the amount of inhibition of NKT activity caused by a biological sample obtained from a subject, or an extract thereof, and comparing the amount of inhibition to a control value, wherein an increase in the amount of inhibition compared to the control value is indicative of a likelihood that the subject suffers from a malignant tumor. In one embodiment, a polar lipid extract is used.

Biological samples suitable for testing in order to carry out the diagnostic method include tissue samples, e.g. whole blood, or fractions thereof such as plasma, serum, and cells, urine, saliva, inter- and intracellular fluid samples, e.g. in particular samples associated with solid tumors such as tumor associated ascites. Means of obtaining suitable biological samples are known to those of skill in the art.

By “likelihood” is meant a statistically significant increase in probability that the subject is afflicted with the disease, disorder or tumor compared to a control population. Preferably the subject is at least a 20%, 50%, 100%, 200%, 500% or 1000% increased risk. Solid tumors include but are not limited to sarcomas, carcinomas, and lymphomas, e.g. ovarian cancer, lung cancer, breast cancer, colon cancer, lymphoma, melanoma and prostate cancer.

Control values can easily be determined by those of skill in the art. For example, a mean value obtained from measuring equivalent biological samples from a normal population of subjects (subjects not known or suspected to be afflicted with a malignant tumor) can be used, with appropriate statistical tests to determine values that will be considered indicative of the presence of a malignant tumor. Persons of skill in the art will appreciate that the presence of a positive result (i.e. indication of malignant tumor) cannot be considered to be an unequivocal indication of the presence of such tumor, as virtually no diagnostic result carries 100% certainty of the result. However, a positive result is expected to be a strong indicator to be considered along with other medical indications to make a diagnosis.

Also provided is a method of treating a malignant tumor such as those described above, by administering a GD3 antagonist, or a compound that inhibits or suppresses the production of GD3, to a subject (e.g. a mammal, especially a human) in need of treatment. Examples of compounds expected to be effective include antibodies, small molecules, siRNA's, and Statins. For example, antibodies or small molecule antagonists to GD3 would be expected to function by suppressing GD3 activity thereby restoring immune function to attack tumor cells, and siRNA's can be designed to suppress the production of GD3. Monoclonal or polyclonal antibodies to GD3 can be prepared by means known in the art. Statins and other commercially available lipid inhibitors such as simvastation (Merck and Co. Inc., Rahway, N.J., USA) and PDMP can block the shedding of inhibitory lipids. While investigators speculate about a potential influence of statins directly on the tumor itself, we hypothesize that, mechanistically, statins work by inhibition of lipid shedding from tumor cells which would otherwise block NKT activation. Another interesting pharmacological agent that has specifically been used to block GD3 synthesis and should be useful in this regard is D-threo-1-phenyl-2-decanoylamino-3-morpholino-1-propanol (PDMP).

Also provided is a method of treating a malignant tumor comprising administering an effective amount of a substance that increases NKT activity by either directly stimulating NKT cell activation or by suppressing production of inhibitors NKT cells made by tumors to a subject in need of treatment.

Dosages of the aforementioned antagonists/compounds can be determined by those of skill in the art without undue experimentation. Dosages are expected to be in the range of 0.1 mg/kg to 1000 mg/kg per day, e.g. 1 mg/kg to 100 mg/kg, depending on route of administration, and the concentration/amount that is delivered to a target site (e.g. a solid tumor). Routes of administration include suitable methods known to those of skill in the art, for example oral; intravenous, intramuscular, subcutaneous, intradermal or intratumor injection, nasal, rectal and other suitable means known to those of skill in the art. Compositions and formulations for administration are known to those of skill in the art. Formulations may include pharmaceutically acceptable diluents, excipients and carriers known to persons of skill in the art as being compatible with the GD3 antagonist, inhibitor or suppressor, and suitable for local or systemic administration to an animal, particularly a human or other mammal, according to the invention. Useful solutions for oral or parenteral administration can be prepared by any of the methods well known in the pharmaceutical arts, described, for example, in Remington's Pharmaceutical Sciences, (Gennaro, A., ed.), Mack Pub., (1990), incorporated herein by reference, in particularly for the description of such diluents, excipients and carriers.

Also provided is a method of screening for anti-cancer agents, comprising contacting a test compound with cells comprising GD3 receptors e.g. NKT cells, NK cells, T cells (Helper cytotoxic and regulatory T cells) and B cells in the presence of GD3, to determine the ability of the compound to suppress or inhibit GD3 binding or activity. Compounds that are able to restore or partially restore the immune function of the cell can be considered as potential anti-cancer agents and subjected to further screening as appropriate.

Also provided is screening method for anti-cancer agents comprising the steps of

a) contacting a test compound with CD1d to obtain treated CD1d;

b) contacting the treated CD1d with NKT cells; and

c) measuring the level of activation of the NKT cells;

wherein increased activation of NKT cells by the treated CD1d compared to a comparable control sample of untreated CD1d is indicative of a potential anti-cancer agent. The steps may be carried out simultaneously or sequentially, in the same or different solutions or mixtures. In one embodiment, the method is carried out in the presence of GD3 or another inhibitor of NKT cell activation.

In another screening method, ascites cells can be cultured with test drugs to inhibit the GD3 production and the culture supernatant tested for its inhibitory activity. Thus, less inhibitory capacity of the supernatant would indicate the presence of a potential anti-cancer drug.

Also provided is a method of modulating the immune system of a subject (e.g. a mammal, in particular a human), by administering an effective amount of GD3 to the subject in order to suppress immune system function. This method may be desirable for treatment of disorders such as autoimmune diseases (e.g. lupus erythematosus, Crohns Disease, Goodpasture's syndrome, rheumatoid arthritis, diabetes mellitus, multiple sclerosis, myasthenia gravis, and anklyosing spondilitis), transplant disorders (e.g. host-graft rejection, graft-host rejection), allergies, and asthma.

Also provided is a method of modulating the immune system of a subject (e.g. a mammal, in particular a human), by administering an effective amount of a GD3 inhibitor or suppressor to the subject in order to stimulate immune system function.

Further provided is a method of monitoring treatment and/or predicting outcome in a subject afflicted with a malignant tumor (e.g. a mammal, in particular a human). In one aspect, the method comprises measuring the level of GD3 in a biological sample obtained from the subject, and comparing the level with the level measured in a comparable sample obtained at a previous time, wherein a decrease in the level of GD3 in the more recent sample is indicative of effective treatment and/or a favorable prognosis. In another aspect, the method comprises measuring the level of inhibition of NKT activity caused by a biological sample obtained from a subject, or an extract thereof, and comparing the level of inhibition to that in a comparable sample obtained at a previous time, wherein a decrease in the level in the more recent sample is indicative of effective treatment and/or a favorable prognosis.

### METHODS

Tumor Associated Ascites. Ovarian cancer associated ascites was collected from patients undergoing primary cytoreductive surgery by the Kelly Gynecologic Oncology Service at Johns Hopkins Medical Institutions. All donors gave written informed consent before enrolling in the study. The Institutional Review Board of Johns Hopkins Medical Institutions approved this investigation.

Cell Lines. Murine L cells transfected with WT cd1d1 cDNA (LCD1dwt) were kindly provided by R. R. Brutkiewicz (Indiana University School of Medicine, Indianapolis, Ind.)(10), and were cultured in DMEM media, supplemented with 2 mM L-glutamine (BioWhittaker), 10% FBS (HyClone), and ciprofloxacin (Serologicals Proteins). TAP (transporter associated with antigen processing)-deficient 174CEM.T2 (T2) cells were maintained in Iscove's modified Dulbecco's media (IMDM), with the same supplements described above.

NKT Cells. The Vα14⁺ NKT cell hybridoma cell lines DN32.D3, (20, 21), N38-2C12, N38-2H4, N38-3C3, and the CD1d1-specific NKT cell hybridoma N37-1A12 (Vα5⁺), have all been described (20, 21, 22). and were cultured in IMDM medium supplemented with 5% FBS and 2 mM L-glutamine. For primary mouse NKT cells, liver mononuclear cells were isolated as described previously (28), then the cells were stained with antibodies against CD3 and NK1.1 (Pharmingen), and sorted using a FACS Aria. Primary human NKT cells were isolated from PBMC using the CD3⁺CD56⁺ isolation kit (Miltenyi).

Generation of artificial Antigen Presenting Cells. The preparation of CD 1 d-Ig based aAPC was performed according to the previously described method (23). The hCD1d-aAPC were loaded with lipid antigen, α-Galactosylceramide (α-GalCer), (5 μg/ml in 1 ml PBS containing 5×10⁷ beads) (Axxora, LLC).

Generation of CMV specific CTL. CMV specific CTL were generated using peptide-pulsed HLA-A2-Ig based aAPCs, as previously described (24).

Treatment of Cells with Tumor Associated Ascites. The ascites was cleared of cellular debris by centrifugation at 250×g for 10 min. The clarified supernatants were then stored at −20° C. For experiments, the supernatants were thawed at 4° C. overnight, then L-vector, L-CDd1wt, CMV-peptide pulsed T2 cells, or aAPC were incubated with ascites (2-5×10⁶ cells per ml of ascites) for 4 h at 37° C., unless otherwise indicated. The cells were subsequently washed three times with 10 ml PBS, resuspended in IMDM medium, supplemented with 5% FBS and 2 mM L-glutamine and co-cultured with or without the indicated NKT hybridomas for 20-24 h at 37° C. The co-cultures with the primary T and NKT cells were incubated for 72 or 48 h, respectively. Cytokine release (IL-2, GM-CSF, or IFN-γ) was measured as an indication of NKT/T cell activation and was measured by standard sandwich ELISA.

Extraction of the polar lipid fraction from the ascites. The polar lipid fraction was isolated from the ascites of the indicated ovarian cancer patients. Methanol then chloroform were added to give chloroform-methanol-water (4:8:3), and the samples were extracted by stirring the mixture at ambient temperature. Insoluble material was removed by centrifugation, and water was then added to the supernatant to give chloroform-methanol-water (4:8:5.6). The resulting phases were separated by centrifugation and the upper phase, containing gangliosides, was desalted using a Sep-Pak C 18 cartridge. The extracted lipids were analyzed by thin-layer chromatography (TLC) (HPTLC Silica Gel 60; Merck, Darmstadt, Germany) with chloroform-methanol-0.25% aqueous KCl (60:35:8) as running solvent. Gangliosides were detected with a resorcinol—HCl Cu2+ reagent. Bovine brain gangliosides were used as standards. The commercially available, purified gangliosides used in the NKT cell assays: Gg3Cer, GM2, GM3, GD3 (Matreya) and GD2 (Calbiochem) were reconstituted in either methanol or chloroform-methanol as suggested by the manufacturer and indicated in the figure legends.

Staining of Human iNKT Cells with hCD1-mIgG Dimers

One microgram of human CD1d-mouse IgG dimer was incubated with 1.4 μg α-GalCer in 50 μL of PBS at 37 ° C. for overnight to load the lipid onto hCD1d-mIgG dimer and used to stain 2×10⁵ Human iNKT cells on ice for 30 min. Then the cells were washed with PBS containing 5% FBS twice and incubated with phycoerythrin-labeled rat anti-mouse IgG1 antibody (A85-1) and APC (allophycocyanin)-labeled anti-human CD3 ε antibody (BD Biosciences, San Diego, Calif., USA) on ice for 30 min. After washing, the stained cells were analyzed with FACSCalibur System (BD Biosciences, San Diego, Calif., USA). Flow cytometry data was analyzed with FlowJo v8.8 software (Tree Star, Inc, Ashland, Oreg.).

### Competitive ELISA Assay

The assay was performed as previously described (REF). In brief, Nunc MaxiSorp flat-bottom 96 well plates (Thermo Fisher Scientific, Waltham, Mass., USA) were coated with 100 μL of Goat anti-mouse IgG Fc gamma antibody (Biomedia, New York, N.Y., USA) (10 μg/mL in 0.1 M NaHCO3, pH 9.6) for overnight at 4° C., and the plates were washed with PBST (PBS containing 0.05% Tween-20) three times and blocked with 1×assay diluent (eBioscience, San Diego, Calif., USA) for 1 h. The plates were washed three times with PBST and 100 μL of lipid-CD1 dimer mixture was added to the plates immediately after washing. The mixture solution was prepared by mixing CD 1 dimer (5 μg/mL) and lipids in the presence of Biotinyl PE (2 μg/mL) in PBS. The CD1-Biotinyl PE complex was detected with HRP-labeled Avidin (eBioscience, San Diego, Calif., USA). Kd of Biotinyl PE was determined by titrating the amount of Biotinyl PE to reach the maximum binding in the absence of competitors and applying the following equation to the data; Y=(Bmax X)/(Kd+X). Kd of Biotinyl PE and Ki of PE and α-GalCer were calculated using GraphPad Prism (Ver. 4.02) (GraphPad Software, Inc., La Jolla, Calif., USA).

### EXAMPLES

### Example 1

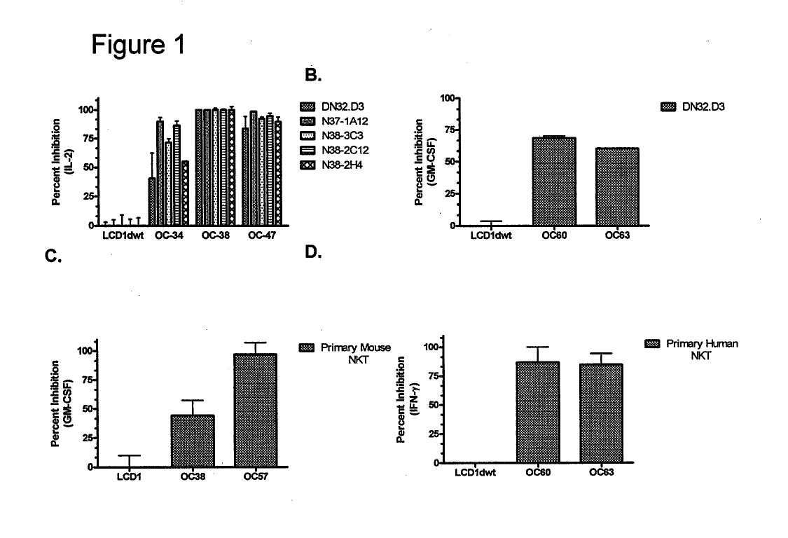

Pretreatment with tumor associated ascites inhibits CD1d-mediated activation of NKT cells. Ascites from a panel of ovarian cancer patients was used to treat CD1d expressing cells (LCD1dwt) and the effect on CD1d mediated antigen presentation was assessed (Table 1). Following treatment with ascites, LCD1dwt cells were washed extensively and co-cultured with NKT cells. We evaluated the ability of the pretreated CD1d expressing cells to stimulate the NKT cells by measuring the IL-2 released in the co-culture supernatants by ELISA. We found that the ability of the CD1d-expressing cells to induce NKT cell cytokine production was reduced by 10−>95% following treatment with the majority of ascites samples examined, as shown in Table 1 and FIG. 1A. Interestingly, we found that antigen presentation to both canonical (Vα14⁺) DN32.D3, N38-3C3, N38-2C12, N38-2H4 and noncanonical (Vα5⁺) N37-1A12 NKT cell hybridomas was inhibited by pretreatment with ascites (FIG. 1A).

TABLE 1

| Code | Diagnosis | Histology | DN32.D3* | N37-1A12* | N38-2C12* |
|---|---|---|---|---|---|
| OC-4 | Ovarian cancer | High-grade serous carcinoma | − | ++ | +++ |
| OC-6 | Ovarian cancer | High-grade serous carcinoma | ++ | +++ | − |
| OC-18 | Ovarian cancer | High-grade serous carcinoma | +++ | ++ | ++ |
| OC-23 | Ovarian cancer | High-grade serous carcinoma | ++ | − | ++ |
| OC-34 | Ovarian cancer | High-grade clear cell carcinoma | +++ | ++++ | ++++ |
| OC-36 | Fallopian tube | High-grade serous carcinoma | ++++ | ++++ | ++ |
| | carcinoma | | | | |
| OC-37 | Ovarian cancer | Mixed clear cell and low grade | − | ++ | ++ |
| | | serous carcinoma | | | |
| OC-38 | Fallopian tube | Moderately differentiated serous | ++++ | ++++ | ++++ |
| | carcinoma | carcinoma | | | |
| OC-40 | Ovarian cancer | High-grade serous and clear cell | ++++ | ++ | +++ |
| | | carcinoma | | | |
| OC-46 | Ovarian cancer | Atypical proliferative (borderline) | ++ | − | ++ |
| | | serous tumor | | | |
| OC-47 | Ovarian cancer | MMMT, carcinosarcoma | ++++ | ++++ | ++++ |
| OC-48 | Ovarian cancer | Well differentiated endometrioid | − | − | ++ |
| | | carcinoma | | | |
| OC-56 | Ovarian cancer | High-grade serous carcinoma | ++++ | ++++ | ++++ |
| OC-57 | Primary peritoneal | High-grade serous carcinoma | ++++ | ++++ | N.D. |
| | cancer | | | | |
| OC-58 | Ovarian cancer | High-grade serous carcinoma | − | − | N.D. |
| OC-59 | Ovarian cancer | High-grade serous carcinoma | ++++ | ++++ | N.D. |
| OC-60 | Ovarian cancer | High-grade serous carcinoma | ++++ | − | N.D. |
| OC-62 | Ovarian cancer | High-grade serous carcinoma | − | +++ | N.D. |
| OC-63 | Ovarian cancer | High-grade adenocarcinoma | ++ | ++ | N.D. |
| OC-64 | Uterine cancer | High-grade serous carcinoma | +++ | ++++ | N.D. |
| OC65 | Ovarian cancer | High-grade serous carcinoma | + | ++++ | N.D. |
| OC66 | Ovarian cancer | High-grade serous carcinoma | ++++ | ++++ | N.D. |
| OC67 | Ovarian cancer | High-grade serous carcinoma | ++++ | + | N.D. |
| OC68 | Ovarian cancer | High-grade endometrioid | ++ | + | N.D. |
| | | carcinoma | | | |
| OC69 | Ovarian cancer | High-grade serous carcinoma | ++ | + | N.D. |
| LP3 | Hepatitis C | | − | − | N.D. |
| LP4 | Hepatitis C | | ++++ | ++++ | N.D. |
| LP5 | Hepatitis C | | − | − | N.D. |
| LP6 | Hepatitis C | | +++ | ++ | N.D. |
| LP7 | Hepatitis C | | − | − | N.D. |
| LP8 | Hepatitis C | | + | ++ | N.D. |

*Percent maximum inhibition of IL-2 production is shown as:
No inhibition = −
<10% = +
10-30 = ++
31-50 = +++
>51% = ++++
N.D. = not determined

### Example 2

Next, we sought to examine whether non-malignant ascites also contained immunosuppressive properties. Thus, we obtained portal hypertension ascites from patients with hepatitis C. As shown in Table 1, we found that half of the ascites ( 3/6) did not inhibit the ability of the CD1d expressing cells to stimulate the NKT cells. In fact, only 1 sample from the patients with hepatitis C produced greater than 50% inhibition. To examine the specificity of inhibition, we analyzed the ability of NKT cells to produce other cytokines. NKT cell hybridomas have been reported to secrete GM-CSF (30), which is important for their maturation and effector functions (31, 32). We found that production of GM-CSF was also suppressed by the majority of the ascites samples tested, as shown with samples OC-60 and OC-63 (FIG. 1B). Ascites treatment of stimulatory cells not only inhibited NKT hybridoma cell lines, but even more importantly it also inhibited the activation of primary mouse NKT cells (FIG. 1C) and primary human NKT cells (FIG. 1D). Taken together, these data demonstrate that ovarian cancer associated ascites can profoundly effect the ability of antigen presenting cells to activate NKT cells.

### Example 3

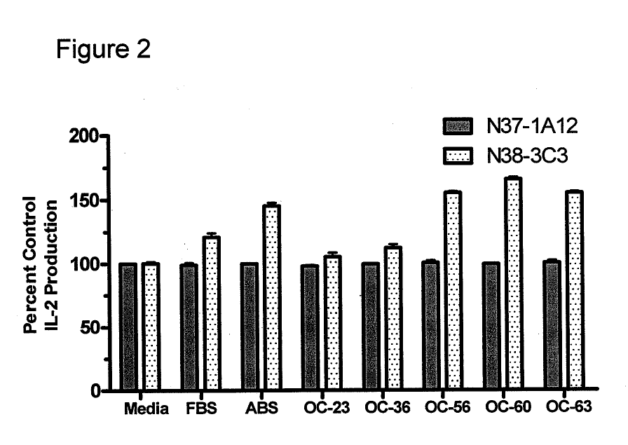

Pretreatment with serum does not inhibit CD1d-mediated activation of NKT cells. Numerous reports have demonstrated that cancer patients have a reduction in NKT cell number and function. As we have found that pretreatment with ovarian cancer associated ascites can reduce the stimulatory capacity of CD1d molecules, it is possible that this effect is not limited locally to the ascites. Therefore, to further analyze if the observed inhibitory effect is limited to the ascites or if it is a more global effect we analyzed the inhibitory capacity of matched serum samples. As shown in FIG. 2, pretreatment with serum from patients or controls (human AB serum and FBS), did not result in decreased stimulation/activation of NKT cells. Moreover, we found that serum pretreatment in some cases even augmented the ability of the antigen presenting cells to induce cytokine production from the NKT cells.

### Example 4

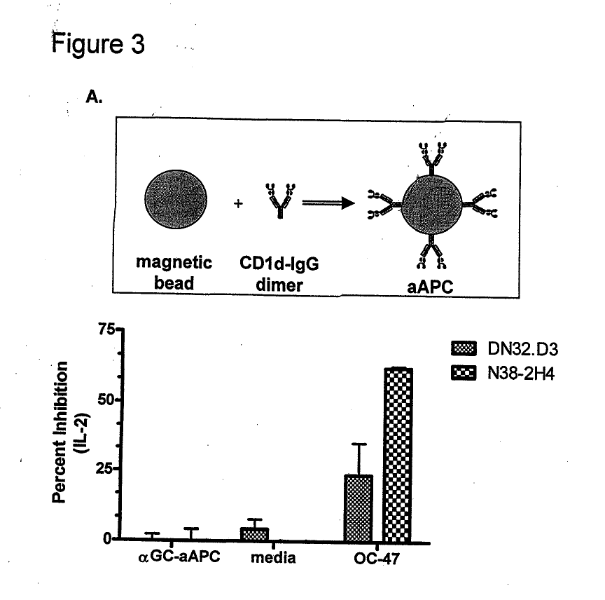

Tumor associated ascites inhibits presentation of α-GalCer by artificial Antigen Presenting cells (aAPC). We developed bead based-CD1d expressing artificial antigen presenting cells (aAPC) (FIG. 3A). The system is based on directly coupling CD1d-Ig to magnetic beads and using them to stimulate the NKT cell hybridomas. Using this cell free tool we can load CD1d-aAPC with alpha-galactosylceramide (α-GalCer), a potent stimulator of NKT cells and determine if antigen processing or editing is necessary for the ascites mediated inhibition. After establishing the system, the α-GalCer loaded aAPC were treated with media or ascites for 4 h, washed extensively, and co-cultured with α-GalCer specific NKT cells. Ascites treatment inhibited α-GalCer presentation by aAPC while mock treatment with media did not affect the aAPC mediated NKT cell activation (FIG. 3B). Thus, these data demonstrate that antigen processing is not necessary for the ascites inhibition. Moreover, the inhibition is not a simply an issue of decreased cell viability.

### Example 5

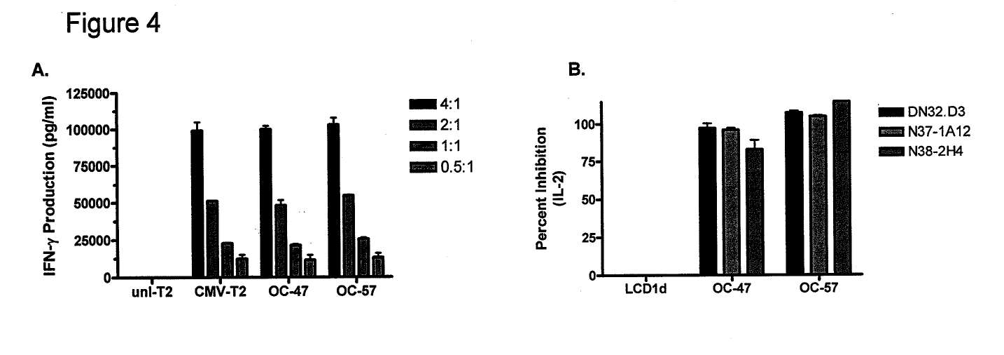

The inhibitory effect mediated by ascites treatment is CD1d-specific. We next examined whether the immunosuppressive effect of ascites was limited to NKT cell activation or if treatment with ascites fluid also inhibited MHC class I mediated activation of CD8⁺ CTL. To address this question, we investigated whether ascites fluid could inhibit the activation of HLA-A2 restricted CMV-specific CTL. In these studies, we used pretreated peptide loaded target cells (T2) to stimulate CMV-specific CD8⁺ T cells and measured IFN-γ release. It is possible that slight alterations in antigen presentation could be masked in the presence of strong stimuli. Therefore, in order to better monitor the effect of treating the target cells with ascites we set up several effector to target cell (E:T) ratios. As shown in FIG. 4A, ascites pretreatment did not inhibit activation of classical CD8⁺ T cells at any ratio examined. In parallel, CD1d-expressing cells were treated with the same ascites samples and their ability to stimulate NKT cells was assessed (FIG. 4B). While there was no effect observed in IFN-γ production by classical CMV-specific CTL, pretreatment with the same ascites samples resulted in an almost complete abrogation of cytokine production by NKT cells. Taken together, these data show that inhibition of antigen presentation by tumor associated ascites is CD1d-specific.

### Example 6

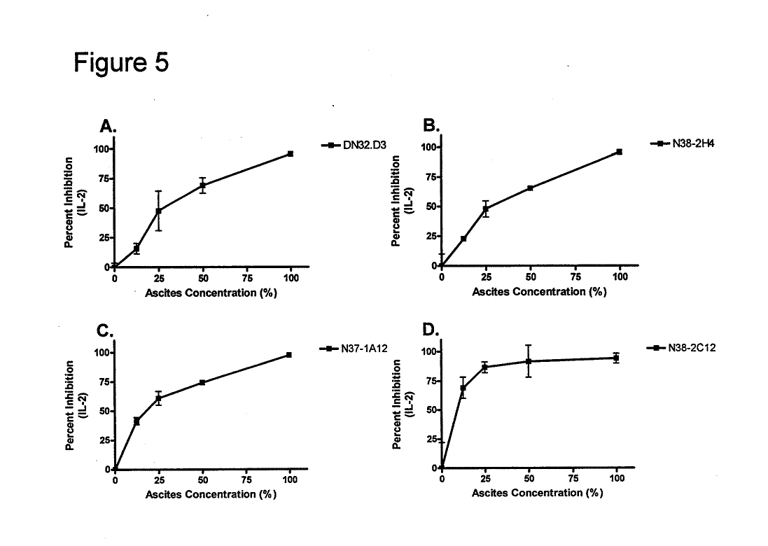

Pretreatment with ascites results in a dose-dependent inhibition of NKT cell activation. Further analysis of the ability of ascites to mediate inhibition of antigen presentation by CD1d was necessary to characterize the effect. We diluted the ascites with cell culture media and treated the LCD1dwt stimulator cells for 4 h, washed extensively and co-cultured them with NKT cells as described above. As shown in FIGS. 5A-D, increasing concentrations of ascites resulted in a concomitant reduction in NKT cell activation, as measured by cytokine release, in all NKT cell hybridomas examined. Even when the antigen presenting cells were treated with 25% ascites fluid or less the blocking effect on NKT cell activation was still observed. While all of the NKT cell lines are CD1d-restricted, they vary in their antigen specificity. Namely, the noncanonical NKT cell hybridoma, N37-1A12, does not recognize α-GalCer in the context of CD1d molecules; however, its activation is similarly reduced in a dose-dependent manner. Interestingly, the dose curves are markedly different for each cell line. In addition, the ascites was passed through 40 micron filters to further characterize its inhibitory effects. There was no change its ability to block CD1d-mediated activation of NKT cells (data not shown). Collectively, these data demonstrate that ovarian cancer associated ascites treatment can block CD1d-mediated activation of NKT cells.

Examples 1-6 demonstrate that treatment of CD1d expressing stimulator cells with ovarian cancer associated ascites inhibited CD1d-mediated activation of NKT cells in vitro. This inhibition resulted in decreased production of multiple cytokines, IL-2, GM-CSF (in murine NKT cells, FIGS. 1A, B, C) and IFN-γ (in primary human NKT cells, FIG. 1D). Interestingly, we found that this effect was limited to the ascites, as it was not found in the following treatment with serum-matched samples from the same patients (FIG. 2).

Ascites treatment of α-GalCer loaded aAPC, impaired their ability to activate NKT cells, which further suggested that the inhibition is independent of antigen processing (FIG. 3). Notably, in these studies we used human CD1d-Ig dimers to generate aAPC. Thus our studies demonstrate an inhibitory effect of ascites fluid on both mouse CD1d molecules (expressed on LCD1d1wt cells), and human CD1d molecules (immobilized on aAPC). Moreover, since αGalCer has been well-characterized as a high affinity ligand and potent stimulator of NKT cells, the level of inhibition observed following treatment with ovarian cancer associated ascites suggests that the putative inhibitory ligand has a very high affinity for CD1d.

To our knowledge this is the first report demonstrating that human tumor ascites specifically suppresses the CD1d/NKT system. Here, we present evidence which demonstrate that ascites, from cancer patients and possibly others, contain inhibitory substances (conceivably lipid antigens) which block CD1d mediated activation of NKT cells. Thus, the presence of activated NKT cells may be a critical prognostic factor for ovarian cancer and more importantly, restoration of their function could be an effective therapeutic strategy.

### Example 7

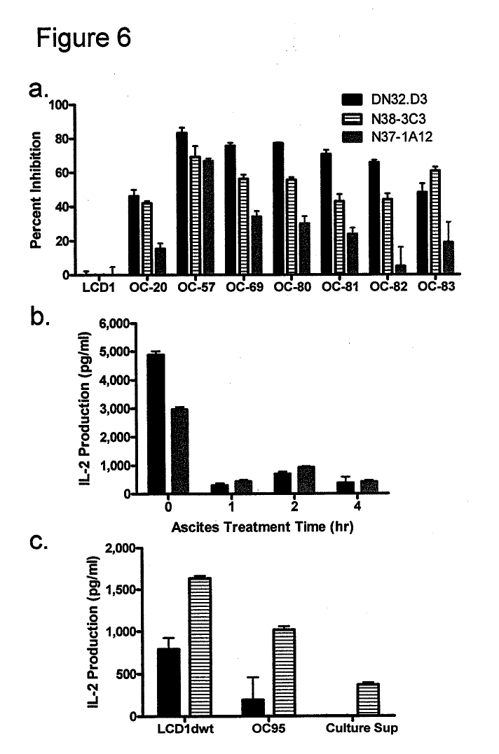

Pretreatment with tumor associated ascites rapidly inhibits CD1d-mediated activation of NKT cells. In this and the following examples, we extend the findings above by evaluating the ability of the pretreated CD1d-expressing cells to stimulate the NKT cells by measuring the IL-2 released in the coculture supernatants by ELISA (FIG. 6). Importantly, we found that treatment with ascites for only 1 hr inhibited the ability of LCD1dwt antigen presenting cells to stimulate NKT cells. In fact, when we treated LCD1dwt antigen presenting cells with cell culture condition media from cells isolated from ascites, we found that antigen presentation to both canonical (Vα14⁺) and noncanonical (Vα5⁺) NKT cell hybridomas (FIG. 6C). These studies support the view that cells present in the ascites are actively producing substances which can abrogate CD1d mediated activation of NKT cells. In order to further characterize the inhibitory effects of ascites, we next tried heat inactivated ascites and found that the ascites maintained its inhibitory effects following heat treatment (data not shown). We then sought to determine whether incubation with ascites treated cells resulted in a permanent functional defect NKT cell activation. In these experiments, fresh untreated stimulator cells were added back to ascites-pretreated cells at the indicated ratios. We found that the addition of untreated cells restored the ability of NKT cells to produce cytokines (data not shown). These data show that stimulation of NKT cells with the pretreated LCD1dwt does not impact on the functionality of the NKT cells but rather simply inhibits NKT cell activation.

### Example 8

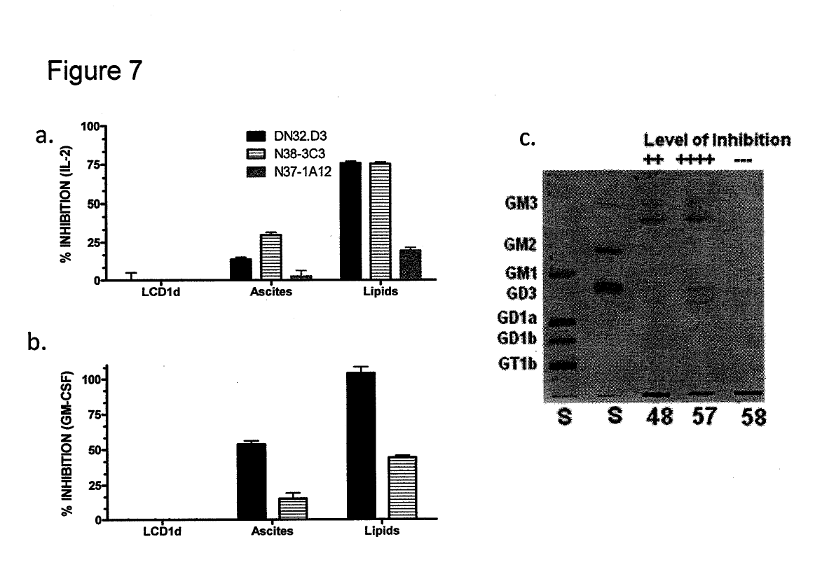

Polar lipids present in ascites inhibit NKT cell recognition of endogenous antigen. In order to identify the substances in the ascites which were mediating inhibition, the polar lipid fraction was extracted. The extracted polar lipid fraction was resuspended in media, and its effects on LCD1dwt antigen presenting cell mediated-activation of NKT cells was analyzed. Pretreatment with the polar lipid fraction significantly reduced the ability of the APC to stimulate NKT cells, as measured by cytokine production (FIGS. 7A & B). In fact, the reduction in CD1d-mediated NKT cell activation following incubation with the polar lipid fraction was equal to or greater than the inhibition observed following ascites pretreatment. Thus, the polar lipid fraction of ovarian cancer associated ascites contains lipids which block NKT cell activation.

As we have found that the inhibitory factor(s) are present in the polar lipid fraction of the ascites, we next analyzed the polar lipid fraction by TLC to determine its ganglioside composition. The majority of patient samples examined contained GM3, GD3, and GT1b; however only GD3 expression appeared to directly correlate with the level of NKT cell inhibition (FIG. 7C).

### Example 9

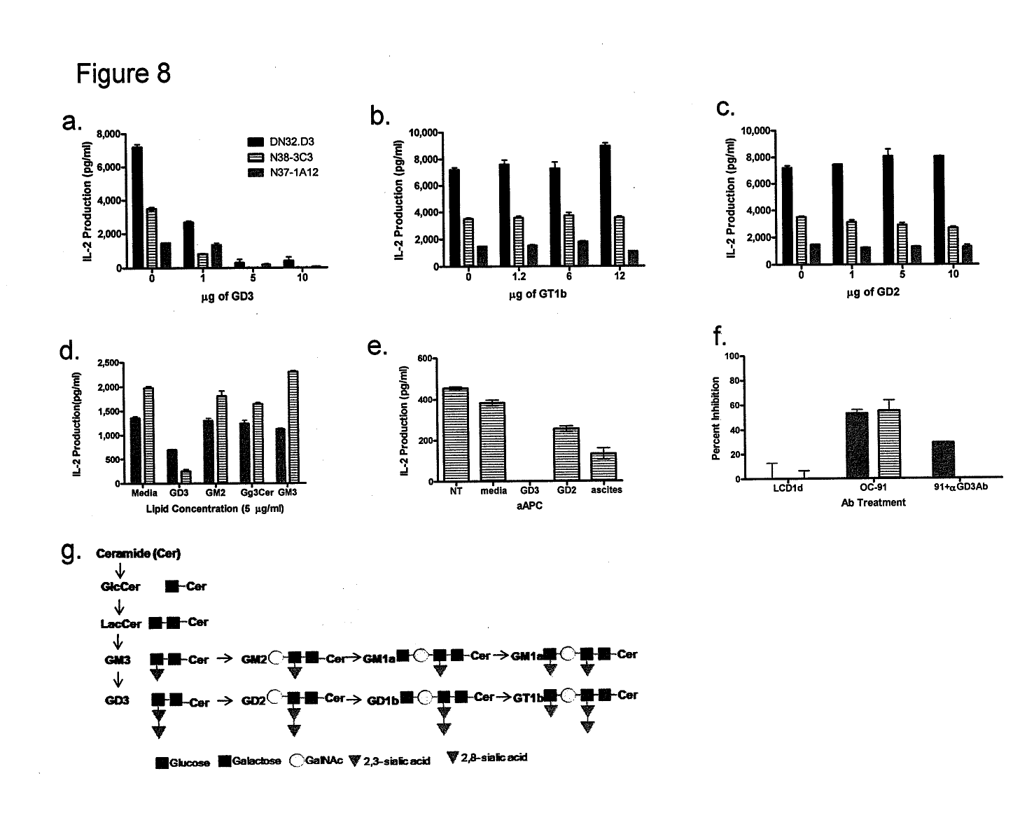

Identification of GD3 as an inhibitory lipid in ovarian cancer ascites. Pretreatment with GD3 results in a dose-dependent inhibition of NKT cell activation. Since ovarian cancer patient ascites is rich in gangliosides, such as GD3 and GD2², we hypothesized that one of these is responsible for the inhibition of NKT cell activation we observed. Therefore, antigen presenting cells were incubated with increasing doses of purified commercially available GD3 (Matreya; GD3 isolated from buttermilk) (FIG. 8A), GD2 (FIG. 8B), and GT1b (FIG. 8C), washed and cocultured with NKT cells. Only GD3 inhibited CD1d-mediated activation of NKT cells in a dose-dependent fashion, while the related gangliosides GD2 and GT1b did not inhibit CD1d-mediated NKT cell activation. In addition, other tumor associated gangliosides, specifically GM3 immediately upstream of GD3 and Gg3Cer have been reported to inhibit canonical NKT cell activation, when used at high concentrations. We pretreated LCD1dwt cells with a low concentration of various purified lipids resuspended in media and assessed their ability to present endogenous antigen to canonical and non-canonical NKT cells (FIG. 8D). Under these conditions with treatment of lipids at a low concentration, it was found that only treatment with GD3 resulted in a significant reduction in NKT cell activation.

### Example 10

In order to determine whether the inhibition observed following pretreatment with GD3 required antigen processing or editing we used bead based-CD1d expressing artificial antigen presenting cells (aAPC). The system is based on directly coupling CD1d-Ig to magnetic beads and using them to stimulate NKT cell hybridomas. Utilizing this unique system, the aAPC were first loaded with alpha-galactosylceramide (α-GalCer), a potent stimulator of NKT cells and then treated with media, ascites, or purified gangliosides for 4 h, washed extensively, and cocultured with canonical NKT cells. Treatment with ascites or GD3 inhibited α-GalCer presentation by aAPC while mock treatment with media or with a control ganglioside, GD2, did not a significant affect aAPC mediated NKT cell activation (FIG. 8E). Taken together, these data demonstrate that antigen processing is not necessary for the GD3 mediated inhibition. To further demonstrate that the presence of GD3 in ovarian cancer associated ascites was responsible for the loss of NKT cell activation, we pretreated ascites with a monoclonal Ab specific for GD3. As shown in FIG. 8F, the presence of antibodies against GD3 restored NKT cell activation, as measured by cytokine release. A schematic diagram of ganglioside biosynthesis is illustrated in FIG. 8g. Next we extracted lipids from a panel of ascites samples and determined the relative concentration of GD3 per militer of ascites (data not shown). We found that all of the tumor associated ascites which inhibit NKT cell activation contain the ganglioside GD3.

### Example 11

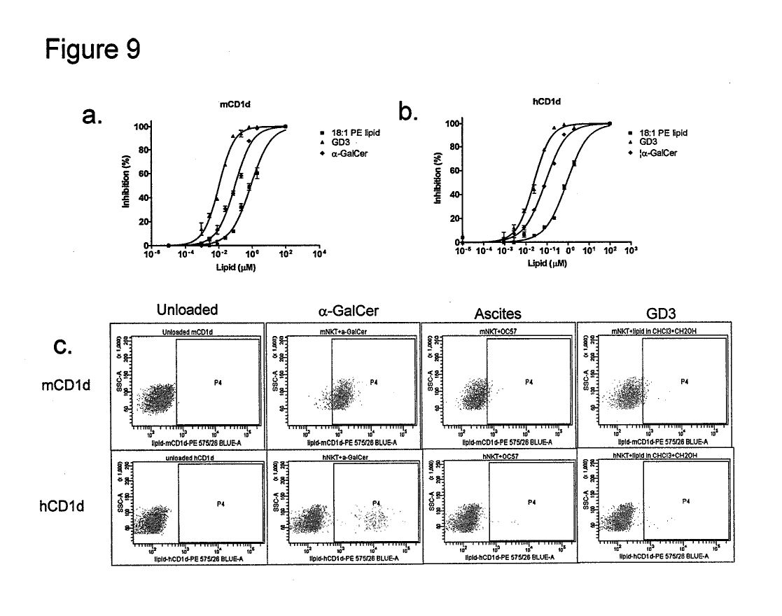

GD3 can bind with high affinity to CD1d molecules. To determine the binding affinity of GD3 to CD1d molecules competition assays were performed. As previously reported (4), this is a competitive binding assay using a tagged lipid, 18:1 Biotinyl PE. As shown in FIGS. 9A & B, a dose dependent competition by GD3 to PE was observed for both human and mouse CD1d molecules. The IC50 shows that purified GD3 binds with high affinity to CD compared to the control, PE or even the potent stimulator a-GalCer. Interestingly, compounds in the ascites, presumably GD3, also had a high affinity for CD1d. As this assay has been shown to reflect lipid-selectivity by CD1 molecules, these data indicate that GD3, as well as possibly other substances in the ascites bind strongly to CD1d molecules.

### Example 12

We next examined whether CD1d-Ig dimers loaded with GD3 or incubated with ascites would bind to NKT cells FIG. 9C. While the controls, α-GalCer loaded human and mouse dimers, were able to bind to NKT cells, CD1d-Ig dimers loaded with GD3 or ascites were not able to stain NKT cells. Thus, these data may explain the inhibitory effect mediated by ascites treatment. GD3 present in the ascites can compete with stimulatory ligands for binding to CD1d molecules. However, these gangliosides loaded into CD1d molecules do not bind to or activate NKT cells.

### Discussion

Previous studies have elucidated roles for specific T cells subsets in epithelial ovarian cancer (EOC) (5-9). As shown in examples 1-5, ovarian cancers and related tumors shed inhibitory substances into the microenviroment which specifically block CD1d mediated antigen presentation to NKT cells. Examples 6-12 demonstrate that at least one mechanism by which tumor associated ascites inhibits NKT cell activation is due to expression of the ganglioside GD3.

Treatment of stimulator cells with ovarian cancer associated ascites results in the specific inhibition of CD1d-mediated activation of NKT cells in vitro. Ascites treatment even inhibited the ability of the GalCer loaded aAPC to stimulate NKT cells. Since α-GalCer has been well-characterized as a high affinity ligand and potent stimulator of NKT cells, the level of inhibition that is observed following treatment with ovarian cancer associated ascites suggested that the putative inhibitory ligand has a very high affinity for CD1d. Therefore, in the current studies we sought for CD1d binding ligands present in the ascites and identified the ganglioside GD3 as a high affinity competitive ligand that inhibits the activation of NKT cells in vitro.

Using a quantitative binding assay, we were able to measure the affinity of the GD3 for CD1 molecules. Surprisingly, the affinity of GD3 was significantly higher than even the affinity of α-GalCer for CD1d. In contrast, most other naturally occurring ligands such as isoglobotrihexosylceramide (iGb3) are thought to be relatively low affinity ligands for CD1d as determined by functional assay. Even glycolipids isolated from foreign pathogenic bacteria such as Sphingomonas are not as active as a-GalCer. Thus our binding studies demonstrating that GD3 has a very high affinity for CD1d, enabling it to displace most NKT activating ligands puts it in a unique category as an immunomodulator.

In conclusion, the data presented herein demonstrates that ovarian cancer ascites contains cells that shed gangliosides, GD3 and possibly others, into the microenvironment which specifically block CD1d mediated antigen presentation to NKT cells. Our current findings demonstrate that treatment of CD1d expressing cells with ascites from ovarian cancer patients inhibits their ability to stimulate NKT cells in vitro. Thus, these data support the view that the presence of GD3 and its impact on the activation and presence of NKT cells can be a critical prognostic factor for ovarian cancer and that restoration of NKT cell function can be an effective therapeutic strategy.

While specific examples have been provided, the above description is illustrative and not restrictive. Any one or more of the features of the previously described embodiments can be combined in any manner with one or more features of any other embodiments in the present invention. Furthermore, many variations of the invention become apparent to those skilled in the art upon review of the specification. The scope of the invention should, therefore, be determined not with reference to the description herein, but instead should be determined with reference to the appended claims along with their full scope of equivalents.

### REFERENCES

1. Chang, X. H. et al. Screening for genes associated with ovarian cancer prognosis. Chin Med J (Engl) 122, 1167-72 (2009).

2. Santin, A. D. et al. Increased levels of gangliosides in the plasma and ascitic fluid of patients with advanced ovarian cancer. Bjog 111, 613-8 (2004).

3. Valentino, L. et al. Shed tumor gangliosides and progression of human neuroblastoma. Blood 75, 1564-7 (1990).

4. Shiratsuchi, T., Schneck, J., Kawamura, A. & Tsuji, M. Human CD1 dimeric proteins as indispensable tools for research on CD1-binding lipids and CD1-restricted T cells. J Immunol Methods 345, 49-59 (2009).

5. Zhang, L. et al. Intratumoral T cells, recurrence, and survival in epithelial ovarian cancer. N Engl J Med 348, 203-13 (2003).

6. Curiel, T. J. et al. Specific recruitment of regulatory T cells in ovarian carcinoma fosters immune privilege and predicts reduced survival. Nat Med 10, 942-9 (2004).

7. Sato, E. et al. Intraepithelial CD8+ tumor-infiltrating lymphocytes and a high CD8+/regulatory T cell ratio are associated with favorable prognosis in ovarian cancer. Proc Natl Acad Sci USA 102, 18538-43 (2005).

8. Ramakrishna, V. et al. Naturally occurring peptides associated with HLA-A2 in ovarian cancer cell lines identified by mass spectrometry are targets of HLA-A2-restricted cytotoxic T cells. Int Immunol 15, 751-63 (2003).

9. Santin, A. D. et al. Induction of ovarian tumor-specific CD8+ cytotoxic T lymphocytes by acid-eluted peptide-pulsed autologous dendritic cells. Obstet Gynecol 96, 422-30 (2000).

10. Sriram, V. et al. Inhibition of glycolipid shedding rescues recognition of a CD1+ T cell lymphoma by natural killer T (NKT) cells. Proc Natl Acad Sci USA 99, 8197-202 (2002).

11. Zeng, G. et al. Identification of CD4+ T cell epitopes from NY-ESO-1 presented by HLA-DR molecules. J Immunol 165, 1153-9 (2000).

12. McKallip, R., Li, R. & Ladisch, S. Tumor gangliosides inhibit the tumor-specific immune response. J Immunol 163, 3718-3726 (1999).

13. Li, R. & Ladisch, S. Abrogation of shedding of immunosuppressive neuroblastoma gangliosides. Cancer Res 56, 4602-4605 (1996).

14. Deng, W., Li, R. & Ladisch, S. Influence of cellular ganglioside depletion on tumor formation. J Natl Cancer Inst 92, 912-917 (2000).

15. Saqr, H. E., Pearl, D. K. & Yates, A. J. A review and predictive models of ganglioside uptake by biological membranes. J Neurochem 61, 395-411 (1993).

16. Ladisch, S., Li, R. & Olson, E. Ceramide structure predicts tumor ganglioside immunosuppressive activity. Proc Natl Acad Sci USA 91, 1974-8 (1994).

17. Ferlito, M., Irani, K., Faraday, N. & Lowenstein, C. J. Nitric oxide inhibits exocytosis of cytolytic granules from lymphokine-activated killer cells. Proc Natl Acad Sci USA (2006).

18. Wu, D. Y., Segal, N. H., Sidobre, S., Kronenberg, M. & Chapman, P. B. Cross-presentation of disialoganglioside GD3 to natural killer T cells. J Exp Med 198, 173-81 (2003).

19. Park, J. E. et al. Fine specificity of natural killer T cells against GD3 ganglioside and identification of GM3 as an inhibitory natural killer T-cell ligand. Immunology 123, 145-55 (2008).

20. Brutkiewicz, R. R., Bennink, J. R., Yewdell, J. W. & Bendelac, A. TAP-independent, beta 2-microglobulin-dependent surface expression of functional mouse CD1.1. J Exp Med 182, 1913-9 (1995).

21. Lantz, O. & Bendelac, A. An invariant T cell receptor alpha chain is used by a unique subset of major histocompatibility complex class I-specific CD4+ and CD4-8-T cells in mice and humans. J Exp Med 180, 1097-106 (1994).

22. Burdin, N. et al. Selective ability of mouse CD1 to present glycolipids: alpha-galactosylceramide specifically stimulates V alpha 14+ NK T lymphocytes. J Immunol 161, 3271-81 (1998).

23. Webb, T. J., Bieler, J. G., Schneck, J. P. & Oelke, M. Ex vivo induction and expansion of natural killer T cells by CD1d1-Ig coated artificial antigen presenting cells. J Immunol Methods 346, 38-44 (2009).

24. Schutz C, Fleck M, Mackensen A, et al. Killer artificial antigen-presenting cells: a novel strategy to delete specific T cells. Blood 2008;111: 3546-52.

We claim:

1. A method of diagnosis of a malignant tumor comprising measuring the level of GD3 in a biological sample obtained from a subject, and comparing the level to a control value, wherein an increase in the level of GD3 compared to the control value is indicative of a likelihood that the subject suffers from a malignant tumor.

2. The method of claim 1 wherein the malignant tumor is a solid tumor.

3. The method of claim 2, wherein the solid tumor is selected from ovarian cancer, lung cancer, breast cancer, colon cancer, lymphoma, melanoma and prostate cancer.

4. The method of claim 1 wherein the subject is a mammal.

5. The method of claim 4 wherein the mammal is a human.

6. The method of claim 1 wherein the biological sample is selected from ascites, peripheral blood, and urine.

7. The method of claim 6 wherein the biological sample is tumor associated ascites.

8. A method of treating a malignant tumor comprising administering an effective amount of a GD3 antagonist or antibody to GD3, or a compound that inhibits or suppresses the production of GD3, to a subject in need of treatment.

9. The method of claim 8 wherein the antagonist, antibody or compound is selected from the group consisting of an antibody, siRNA, glycolipid synthesis inhibitor and a statin.

10. The method of claim 9 wherein a monoclonal or polyclonal antibody is administered.

11. The method of claim 8 wherein the malignant tumor is a solid tumor.

12. The method of claim 9, wherein the solid tumor is selected from ovarian cancer, lung cancer, breast cancer, colon cancer, lymphoma, melanoma and prostate cancer.

13. The method of claim 8 wherein the subject is a mammal.

14. The method of claim 13 wherein the mammal is a human.

15. A method of screening for an anti-cancer agent, comprising contacting a test compound with test cells comprising GD3 receptors in the presence of GD3, and comparing GD3 binding or an immune activity of the test cells to GD3 binding or the immune activity of control cells in which GD3 but no test compound is present, wherein a decrease in GD3 binding or increase in immune activity of the test cells compared to the control cells is indicative of a potential anti-cancer agent.

16. The method of claim 15 wherein the test cells are NKT cells.

17. A method of screening for an anti-cancer agent, comprising the steps of
a) contacting a test compound and GD3 with CD1d to obtain treated CD1d;
b) contacting the treated CD1d with NKT cells; and
c) measuring the level of activation of the NKT cells;
wherein increased activation of NKT cells by the treated CD1d compared to a comparable control sample of untreated CD1d is indicative of a potential anti-cancer agent.

18. The method of claim 17 in which steps (a-c) are carried out in the same mixture.

19. The method of claim 17 wherein steps (a-c) are carried out sequentially.

20. A method of modulating the immune system of a subject, comprising administering an effective amount of GD3 to the subject in order to suppress immune system function.

21. The method of claim 20 which is a method for treating an autoimmune disease, host versus-graft rejection, graft versus-host rejection, an allergy, or asthma.

22. The method of claim 21 wherein the autoimmunine disease is selected from the group consisting of lupus erythematosus, Crohns Disease, Goodpasture's syndrome, rheumatoid arthritis, diabetes mellitus, multiple sclerosis, myasthenia gravis, and anklyosing spondilitis.

23. A method of monitoring treatment and/or predicting outcome in a subject afflicted with a malignant tumor comprising measuring the level of GD3 in a biological sample obtained from the subject, and comparing the level with the level measured in a comparable sample obtained at a previous time, wherein a decrease in the level of GD3 in the more recent sample is indicative of effective treatment and/or a favorable prognosis.

24. The method of claim 23 wherein the malignant tumor is a solid tumor.

25. The method of claim 24, wherein the solid tumor is selected from ovarian cancer, lung cancer, breast cancer, colon cancer, lymphoma, melanoma and prostate cancer.

26. The method of claim 23 wherein the subject is a mammal.

27. The method of claim 26 wherein the mammal is a human.

28. The method of claim 26 wherein the biological sample is selected from ascites, peripheral blood, and urine.

29. The method of claim 28 wherein the biological sample is tumor associated ascites.

30. A method of monitoring treatment and/or predicting outcome in a subject afflicted with a malignant tumor comprising measuring level of inhibition of NKT activity caused by a biological sample obtained from a subject, or an extract thereof, and comparing the level of inhibition to that in a comparable sample obtained at a previous time, wherein a decrease in the level in the more recent sample is indicative of effective treatment and/or a favorable prognosis.

31. The method of claim 30 wherein the malignant tumor is a solid tumor.

32. The method of claim 31, wherein the solid tumor is selected from ovarian cancer, lung cancer, breast cancer, colon cancer, lymphoma, melanoma and prostate cancer.

33. The method of claim 32 wherein the subject is a mammal.

34. The method of claim 33 wherein the mammal is a human.

35. The method of claim 30 wherein the biological sample or extract is selected from ascites, peripheral blood, and urine.

36. The method of claim 35 wherein the biological sample or extract is tumor associated ascites.

37. A method of diagnosis of a malignant tumor comprising measuring the amount of inhibition of NKT activity caused by a biological sample obtained from a subject, or an extract thereof, and comparing the amount of inhibition to a control value, wherein an increase in the amount of inhibition compared to the control value is indicative of a likelihood that the subject suffers from a malignant tumor.

38. The method of claim 37 wherein the biological sample is a polar lipid extract.

39. The method of claim 37 wherein the inhibition is caused by GD3.

40. The method of claim 37 wherein the malignant tumor is a solid tumor.

41. The method of claim 40, wherein the solid tumor is selected from ovarian cancer, lung cancer, breast cancer, colon cancer, lymphoma, melanoma and prostate cancer.

42. The method of claim 37 wherein the subject is a mammal.

43. The method of claim 42 wherein the mammal is a human.

44. The method of claim 37 wherein the biological sample is selected from ascites, peripheral blood, and urine.

45. The method of claim 44 wherein the biological sample is tumor associated ascites.

46. A method of screening for an anti-cancer agent, comprising culturing ascites cells and/or tumor cells with a test drug to inhibit GD3 production, and measuring the inhibitory activity of the culture supernatant on NKT cells or other immune cells, wherein a reduction in inhibitory activity of the supernatant from the culture with the test drug compared to a control culture without the test drug is indicative of the presence of a potential anti-cancer drug.

47. A method of treating a malignant tumor comprising administering an effective amount of a substance that increases NKT activity by either directly stimulating NKT cell activation or by suppressing production of inhibitors of NKT cells made by tumors to a subject in need of treatment.

48. The method of claim 47 wherein the malignant tumor is a solid tumor.

49. The method of claim 48, wherein the solid tumor is selected from ovarian cancer, lung cancer, breast cancer, colon cancer, lymphoma, melanoma and prostate cancer.

50. The method of claim 47 wherein the subject is a mammal.

51. The method of claim 50 wherein the mammal is a human.

52. The method of claim 47 wherein the biological sample is selected from ascites, peripheral blood, and urine.

53. The method of claim 52 wherein the biological sample is tumor associated ascites.
